(19) United States
(12) Patent Application Publication
(10) Pub. No.: US 2024/0315625 A1
(43) Pub. Date: Sep. 26, 2024

(54) Blood Draw Syringe With Hemolysis Protection

(71) Applicant: Becton, Dickinson and Company, Franklin Lakes, NJ (US)

(72) Inventors: Yiping Ma, Layton, UT (US); Jordan Ciciliano, Durham, NC (US); Jonathan Karl Burkholz, Salt Lake City, UT (US)

(21) Appl. No.: 18/733,310

(22) Filed: Jun. 4, 2024

Related U.S. Application Data
(63) Continuation of application No. 17/496,858, filed on Oct. 8, 2021, now Pat. No. 12,023,159.
(60) Provisional application No. 63/090,602, filed on Oct. 12, 2020.

(51) Int. Cl. A61B 5/15

(52) U.S. Cl. CPC A61B 5/150992; A61B 5/150236

(57) ABSTRACT

A blood collection device may include a syringe with hemolysis protection. The syringe with hemolysis protection may be coupled to a catheter assembly and used for blood draw. The blood collection device may include the syringe, which may include a distal end, and a flow restrictor. The flow restrictor may include a distal end and a proximal end. The proximal end of the flow restrictor may be coupled to the distal end of the syringe, which may protect against hemolysis.

### CROSS-REFERENCE TO RELATED APPLICATION

The present application is a continuation of U.S. patent application Ser. No. 17/496,858, entitled “Blood Draw Syringe with Hemolysis Protection”, filed Oct. 8, 2021, which claims priority to U.S. Provisional Application Ser. No. 63/090,602, entitled “Blood Draw Syringe with Hemolysis Protection”, filed Oct. 12, 2020, the entire disclosures of each of which are hereby incorporated by reference in their entirety.

### BACKGROUND

A catheter is commonly used to infuse fluids into vasculature of a patient. For example, the catheter may be used for infusing normal saline solution, various medicaments, or total parenteral nutrition.

The catheter may include a peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may remove the introducer needle, leaving the catheter in place for future fluid infusion.

Blood draws through peripheral intravenous catheters are not routinely performed, in large part due to a risk of hemolysis of a blood sample withdrawn through the peripheral intravenous catheter. When blood draw is performed through the peripheral intravenous catheter, a commonly used blood collection container is a VACUTAINER® blood collection tube, available from Becton Dickinson & Company. In some instances, a syringe may instead be used. However, the syringe has not been shown to reduce a risk of hemolysis compared to the VACUTAINER®.

Unfortunately, as blood is drawn into the syringe or VACUTAINER®, red blood cells are in a high shear stress state and susceptible to hemolysis due to a high pressure differential between the vein and the syringe or VACUTAINER®. Hemolysis may result in rejection and discard of a blood sample. The high pressure differential can also result in catheter tip collapse, vein collapse, or other complications.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure generally relates to a blood collection device and related devices, systems, and methods. In some embodiments, a blood collection system may include a catheter assembly, which may include a catheter adapter and a catheter extending distally from the catheter adapter. In some embodiments, the blood collection system may include the blood collection device coupled to the catheter assembly. In some embodiments, the blood collection device may include a syringe, which may include a distal end. In some embodiments, the blood collection device may include a flow restrictor, which may include a distal end and a proximal end. In some embodiments, the proximal end of the flow restrictor may be coupled to the distal end of the syringe.

In some embodiments, the catheter assembly may include an extension tube. In some embodiments, a distal end of the extension tube may be integrated with the catheter adapter. In some embodiments, the blood collection device may be coupled to a proximal end of the extension tube.

In some embodiments, the distal end of the syringe may include a first luer adapter. In some embodiments, the proximal end of the flow restrictor may include a second luer adapter coupled to the first luer adapter. In some embodiments, the flow restrictor may be monolithically formed as a single unit. In these and other embodiments, the distal end of the flow restrictor may include a third luer adapter.

In some embodiments, the distal end of the flow restrictor may include the third luer adapter. In these and other embodiments, the flow restrictor may include an extension tube disposed between the second luer adapter and the third luer adapter. In these and other embodiments, the flow restrictor may include a first piece monolithically formed as a single unit and a second piece monolithically formed as a single unit. In some embodiments, a proximal end of the extension tube may be integrated within the first piece. In some embodiments, a distal end of the extension tube may be integrated within the second piece. In some embodiments, the first piece may include the second luer adapter. In some embodiments, the second piece may include the third luer adapter.

In some embodiments, a geometric factor of the flow restrictor is represented by G_{f}. In some embodiments, G_{f} may be 1.43E7 1/in³±30%. In some embodiments, G_{f} may be 3.70E6 1/in³±30%. In some embodiments, G_{f} may be between 1.43E7 and 3.70E6. In some embodiments, G_{f} may be 1.43E7 1/in³±10% or 3.70E6 1/in³±10%.

In some embodiments, the flow restrictor may include a fluid pathway extending through the flow restrictor. In some embodiments, a diameter of the fluid pathway may be uniform along an entirety of a length of the fluid pathway. In these embodiments, the diameter is represented by D, the length is represented by L, and D⁴/L may be 2.7E-7 or 7.0E-8.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example blood collection device that includes hemolysis protection, according to some embodiments;

FIG. 1B is a cross-sectional view of the blood collection device of FIG. 1A, according to some embodiments;

FIG. 2A is an upper perspective view of another example blood collection device that includes hemolysis protection, according to some embodiments;

FIG. 2B is a cross-sectional view of the blood collection device of FIG. 2A, according to some embodiments;

FIG. 3 is an upper perspective view of an example blood collection system, according to some embodiments;

FIG. 4 is a bar graph illustrating blood collection rates with various catheter gauges and flow restrictors under different syringe draw rates, according to some embodiments;

FIG. 5 is a bar graph illustrating maximum shear stress with various catheter gauges and flow restrictors under different syringe draw rates, according to some embodiments; and

FIG. 6 is a graph illustrating plasma free hemoglobin for catheter assemblies with and without a flow restrictor, according to some embodiments.

### DESCRIPTION OF EMBODIMENTS

In some embodiments, a blood collection device 10 may include a syringe 12, which may include a distal end 14 and a proximal end 16. In some embodiments, the syringe 12 may include a sliding plunger 18 that fits tightly in a barrel or tube 20. In some embodiments, the sliding plunger 18 may be pulled proximally with respect to the tube 20, allowing the syringe 12 to draw in a liquid, such as blood, through an orifice 22 at the distal end 14. In some embodiments, the sliding plunger 18 may be pushed distally with respect to the tube 20, allowing the syringe 12 to then expel the liquid through the orifice 22. In some embodiments, the syringe 12 may be configured to hold between about 1 and 20 milliliters of the liquid.

In some embodiments, the blood collection device 10 may include a flow restrictor 24, which may include a distal end 26 and a proximal end 28. In some embodiments, the proximal end 28 of the flow restrictor 24 may be coupled to the distal end 14 of the syringe 12. In some embodiments, the flow restrictor 24 may be removably coupled to the distal end 14 of the syringe 12. In other embodiments, the flow restrictor 24 may be permanently coupled to the distal end 14 of the syringe 12.

In some embodiments, the distal end 14 of the syringe 12 may include a first luer adapter 30. In some embodiments, the proximal end 16 of the flow restrictor 24 may include a second luer adapter 32 coupled to the first luer adapter 30. In some embodiments, the first luer adapter 30 may include a male luer adapter, which may be threaded or engaged in a slip-fit with the second luer adapter 32, which may include a female luer adapter. In some embodiments, a threaded connection between the first luer adapter 30 and the second luer adapter 32 may prevent accidental uncoupling of the syringe 12 from the flow restrictor 24 during blood collection.

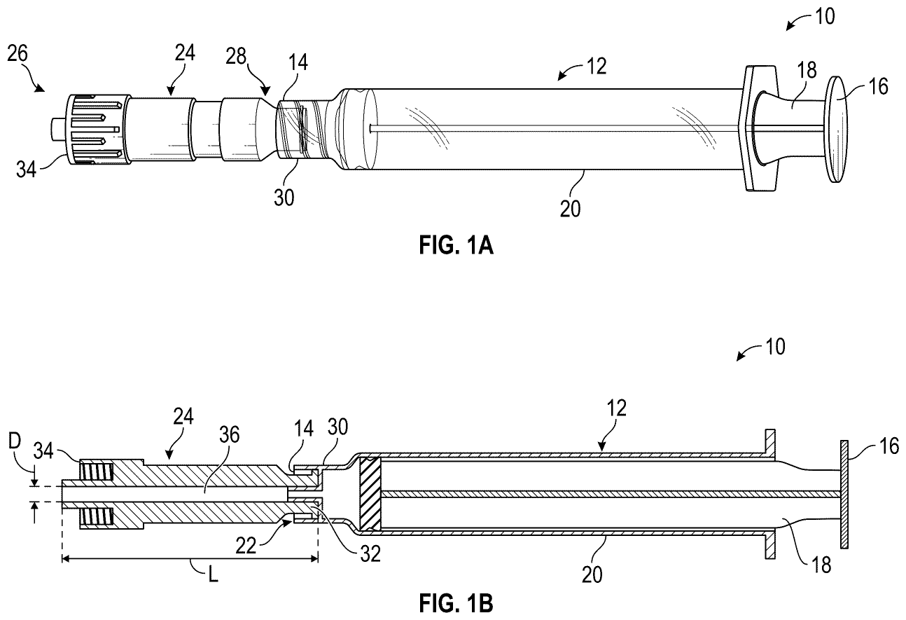

In some embodiments, the flow restrictor 24 may be monolithically formed as a single unit, as illustrated, for example, in FIGS. 1A-1B. In these and other embodiments, the distal end 26 of the flow restrictor 24 may include a third luer adapter 34. In some embodiments, the third luer adapter 34 may include a male luer adapter. In some embodiments, the distal end 26 of the flow restrictor 24 may be configured to couple to a catheter assembly, which may be inserted into vasculature of a patient. In some embodiments, the sliding plunger 18 may be pulled in a proximal direction to withdraw blood from vasculature of the patient into the tube 20 of the syringe 12.

In some embodiments, a length of the flow restrictor 24 may be less than a length of the syringe 12 to facilitate easy handling by a user. In some embodiments, the flow restrictor 24 may include a fluid pathway 36 extending through the flow restrictor 24. Generally, blood cells experience shear stress as blood flows in a fluid pathway. The maximum shear stress is along the wall of the blood cell, or wall shear stress. Wall shear stress on blood cells is considered a major source of hemolysis and mechanical damage to blood cells.

In some embodiments, the flow restrictor 24 may provide hemolysis protection. In further detail, the flow restrictor 24 may limit a maximum blood collection rate, which in turn may limit a maximum shear stress during blood collection and reduce hemolysis. In some embodiments, the flow restrictor 24 may be configured to limit a maximum shear stress that blood cells are exposed to during a syringe draw to a predetermined or target value.

Fluid flow in a flow restrictor with a tubular fluid pathway therethrough can be analyzed using Poiseuille's equation:

$$Q = \frac{π {D}^{4} Δ P}{128 μ L} = \frac{Δ P}{{R}_{f}}$$

where P is a change in pressure gradient across the flow restrictor, D and L are the inner diameter and length, respectively, of the fluid pathway 36 through the flow restrictor, μ is the viscosity of a fluid, and

$${R}_{f} = \frac{128 μ L}{π {D}^{4}}$$

is the fluid resistance. Since y is the viscosity of the fluid and not part of the flow restrictor geometry, a geometric factor G_{f} is defined such that R_{f} (the fluid resistance) is

$${R}_{f} = \frac{128 μ}{π} {G}_{f} ,$$

where

$${G}_{f} = \frac{L}{{D}^{4}} .$$

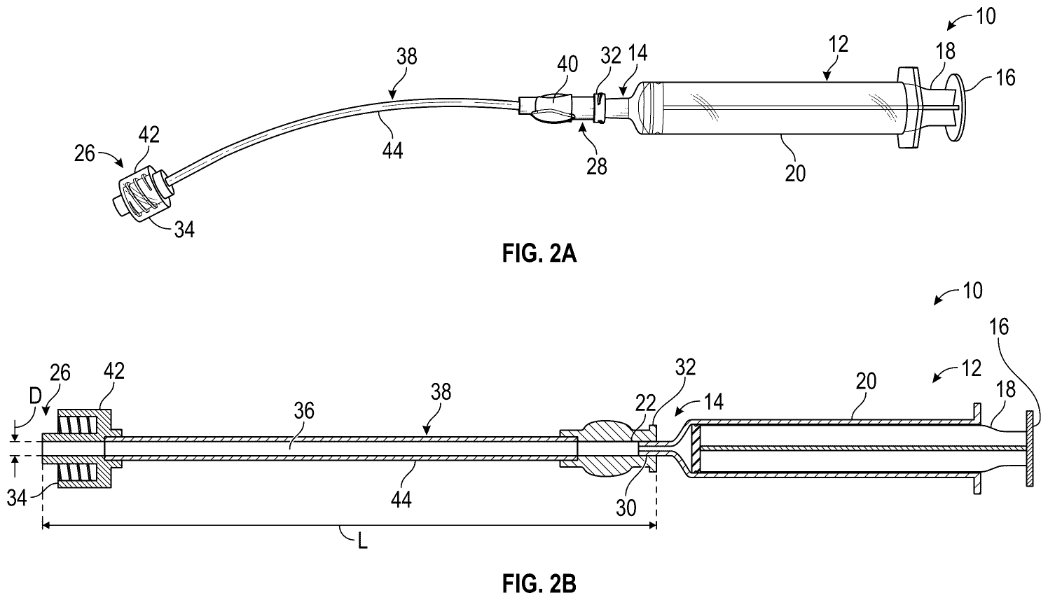

In some embodiments, the flow restrictor may include the flow restrictor 24 or the flow restrictor 38 (see, for example, FIGS. 2A-2B).

In response to the fluid pathway 36 having multiple sections with lengths (L1, L2, L3) and inner diameters of (D1, D2, D3), the fluid resistance is:

$${G}_{f} = \frac{L 1}{D {1}^{4}} + \frac{L 2}{D {2}^{4}} + \frac{L 3}{D {3}^{4}}$$

In some embodiments, the flow restrictor may be configured to limit G_{f}, and thereby limit hemolysis. For example, for the flow restrictor 24, G_{f} may be about 1.43E7 1/in³. In some embodiments, for the flow restrictor 24, G_{f} may be 1.43E7 1/in³±10% or 1.43E7 1/in³±30%.

In some embodiments, an inner diameter of the fluid pathway 36 may be uniform along an entirety of a length of the fluid pathway 36. In some embodiments, D4/L is about 2.7E-7 which may reduce the wall sheer stress to reduce hemolysis when the flow restrictor 24 is used with a 20G catheter. In some embodiments, D⁴/L is about 7.0E-8, which may reduce the wall sheer stress to reduce hemolysis when the flow restrictor 24 is used with a 22G catheter.

Referring now to FIGS. 2A-2B, in some embodiments, the blood collection device 10 may include a flow restrictor 38. In some embodiments, the flow restrictor 38 may be similar or identical to the flow restrictor 24 in terms of one or more features and/or operation. In some embodiments, the flow restrictor 38 may also be configured to limit G_{f}, and thereby limit hemolysis. For example, for the flow restrictor 38, G_{f} may be about 3.70E6 1/in³. In some embodiments, for the flow restrictor 38, G_{f} may be 3.70E6 1/in³±10% or 3.70E6 1/in³±30%. In some embodiments, a particular flow restrictor, such as, for example, the flow restrictor 24 or the flow restrictor 38, may have a G_{f} between 1.43E7 and 3.70E6. In some embodiments, the inner diameter of the fluid pathway 36 may be variable along a length of the fluid pathway 36. In these and other embodiments, the flow restrictor 38 may include multiple sections formed with tubes having different inner diameters that are joined together or a single tube with a varying inner diameter along its length.

In some embodiments, the distal end 26 of the flow restrictor 38 may include the third luer adapter 34. In these and other embodiments, the flow restrictor 38 may include an extension tube 44 disposed between the second luer adapter 32 and the third luer adapter 34. In these and other embodiments, the flow restrictor 38 may include a first piece 40 monolithically formed as a single unit and/or a second piece 42 monolithically formed as a single unit. In some embodiments, a proximal end of the extension tube 44 may be integrated within the first piece 40. In some embodiments, a distal end of the extension tube 44 may be integrated within the second piece 42. In some embodiments, the first piece 40 may include the second luer adapter 32. In some embodiments, the second piece 42 may include the third luer adapter 34.

In some embodiments, a length of the flow restrictor 38 may be less than the length of the syringe 12 to facilitate easy handling by the user. In some embodiments, the flow restrictor 38 may include the fluid pathway 36 extending through the flow restrictor 24. In some embodiments, the inner diameter of the fluid pathway 36 may be uniform along an entirety of the length of the fluid pathway 36. In some embodiments, the inner diameter is represented by D, the length is represented by L, and D⁴/L is 2.7E-7, which may reduce the wall sheer stress to reduce hemolysis when the flow restrictor 38 is used with a 20G catheter. In some embodiments, the inner diameter is represented by D, the length is represented by L, and D⁴/L is 7.0E-8, which may reduce the wall sheer stress to reduce hemolysis when the flow restrictor 38 is used with a 22G catheter.

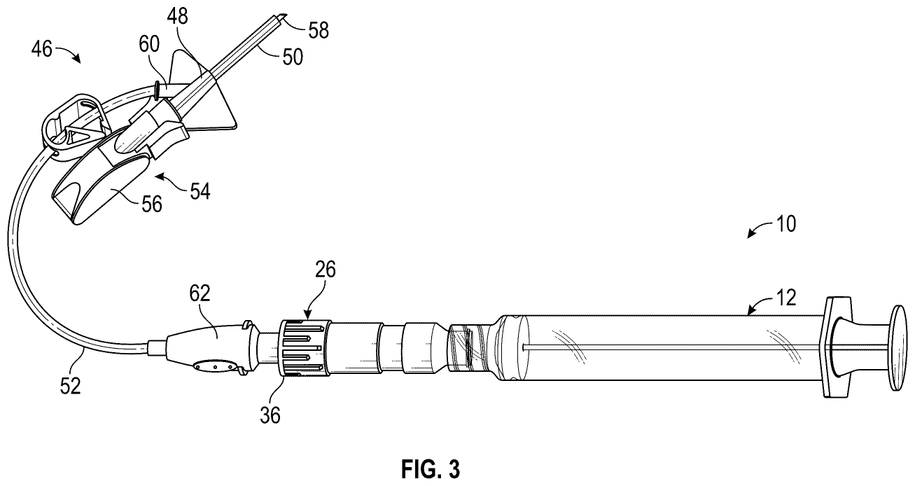

Referring now to FIG. 3, a flow restrictor, such as the flow restrictor 24 (see, for example, FIGS. 1A-1B) or the flow restrictor 38 (see, for example, FIGS. 2A-2B), may be coupled to a catheter assembly 46. In some embodiments, the catheter assembly 46 may include a catheter adapter 48 and a catheter 50 extending distally from the catheter adapter 48. In some embodiments, the catheter assembly 46 may include an extension tube 52. In some embodiments, a distal end of the extension tube 52 may be integrated with the catheter adapter 48. In some embodiments, the blood collection device 10 may be coupled to a proximal end of the extension tube 52.

In some embodiments, a needle assembly 54 may be coupled to the catheter assembly 46. In some embodiments, the needle assembly 54 may include a needle hub 56 and an introducer needle 58 secured within the needle hub 56. In some embodiments, in response to the catheter 50 being inserted into the vasculature of the patient, the needle assembly 54 may be removed from the catheter assembly 46. In some embodiments, the proximal end of the extension tube 52 may be integrated with a side port 60 of the catheter adapter 48.

In some embodiments, the catheter assembly 46 may include or correspond to any suitable catheter assembly, such as, for example, the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable catheter assembly. In some embodiments, the catheter 50 may include a peripheral intravenous catheter (PIVC), a peripherally inserted central catheter (PICC), a midline catheter, or another suitable catheter.

In some embodiments, in response to insertion of the catheter 50 into the vasculature, blood may flow proximally through a fluid pathway of the catheter assembly 46, which may include one or more of the following: the catheter 50, the catheter adapter 48, the extension tube 52, an adapter 62, the flow restrictor, and the syringe 12.

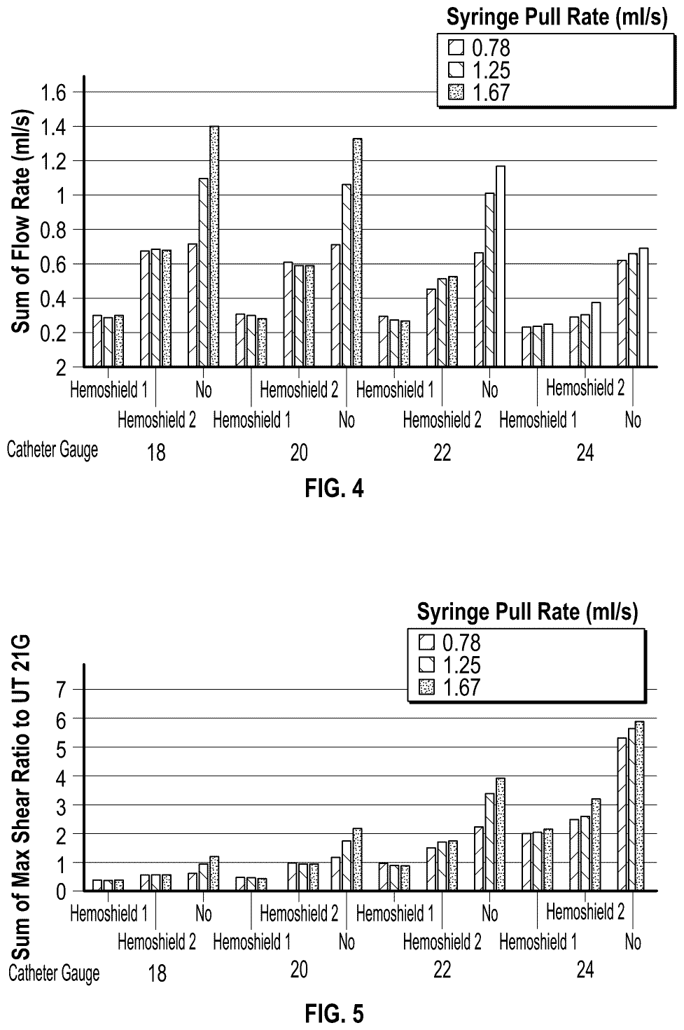

Referring now to FIG. 4, a bar graph illustrates blood collection rates with various catheter gauges and flow restrictors under different syringe draw rates, according to some embodiments. “Hemoshield 1” refers to a first example flow restrictor coupled to a catheter assembly. Hemoshield 1 may include the flow restrictor 24 of FIGS. 1A-1B or the flow restrictor 38 of FIGS. 2A-2B coupled to the catheter assembly. In this example, D\hat{ }4/L for Hemoshield 1 is about 7 e-8, but D\hat{ }4/L may vary. “Hemoshield 2” refers to another example flow restrictor coupled to the catheter assembly. Hemoshield 2 may include the flow restrictor 24 of FIGS. 1A-1B of the flow restrictor 38 of FIGS. 2A-2B coupled to the catheter assembly. In this example, D\hat{ }4/L for Hemoshield 2 is about 2.7 e-8, but D\hat{ }4/L may vary. “No” refers to the catheter assembly that is not coupled to a flow restrictor.

In the absence of a flow restrictor, the blood flow rate varies significantly with the syringe draw rate. However, with the use of the flow restrictors (Hemoshield 1 and Hemoshield 2 in the experiment), the flow rate through the catheter during blood collection becomes independent of the syringe draw rate. This will greatly reduce variation in syringe draws in the clinical setting, according to some embodiments.

FIG. 5 is a bar graph illustrating maximum shear stress with various catheter gauges and flow restrictors under different syringe draw rates, according to some embodiments. Again, “Hemoshield 1” refers to the first example flow restrictor coupled to the catheter assembly. Hemoshield 1 may include the flow restrictor 24 of FIGS. 1A-1B or the flow restrictor 38 of FIGS. 2A-2B coupled to the catheter assembly. In this example, D\hat{ }4/L for Hemoshield 1 is about 7 e-8, but D\hat{ }4/L may vary. “Hemoshield 2” refers to another example flow restrictor coupled to the catheter assembly. Hemoshield 2 may include the flow restrictor 24 of FIGS. 1A-1B of the flow restrictor 38 of FIGS. 2A-2B coupled to the catheter assembly. In this example, D\hat{ }4/L for Hemoshield 2 is about 2.7 e-8, but D\hat{ }4/L may vary. “No” refers to the catheter assembly that is not coupled to a flow restrictor. “UT 21G” refers to a prior art catheter assembly with a 21G catheter.

The resultant max shear stress for each catheter gauge can be shown as a ratio to the max shear stress of a UT 21G with VACUTAINER®, which was previously considered the gold standard for blood draws. As shear stress leads to mechanical hemolysis, reducing the shear stress of catheter draws to that of the UT 21G reduces the risk of hemolysis. The data shows that for an 18G catheter, the risk of hemolysis is low when using a syringe to draw blood. However, as the catheter's gauge increases, the risk of hemolysis also increases. For a 20G catheter, the risk of hemolysis is moderate at low syringe pull rates, but increases at higher pull rates. With a flow restrictor with D\hat{ }4/L of 2.7 e-7 the max shear with a catheter draw is reduced to the max shear of 21G UT for all tested syringe draw rates. For a 22G catheter, the risk of hemolysis is significant at higher draw rates. A flow restrictor with D\hat{ }4/L of 7 e-8 again reduces the shear rate to that of the gold standard for all tested syringe pull rates. For a 24G catheter, the tested flow restrictors are able to reduce the max shear stress 2.5 to 3× depending on the syringe draw rate.

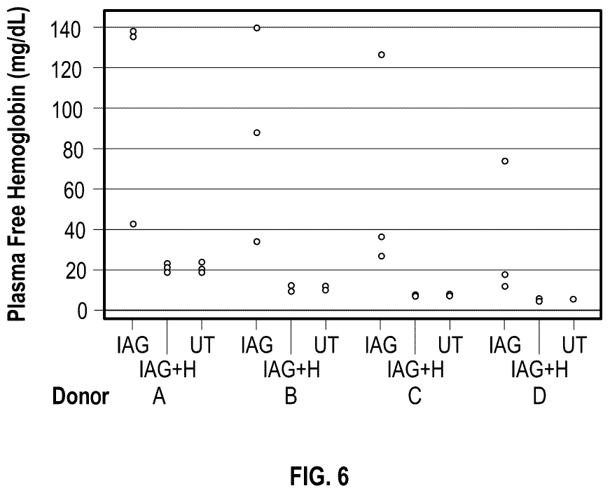

FIG. 6 is a graph illustrating plasma free hemoglobin for catheter assemblies with and without a flow restrictor, according to some embodiments. “IAG” refers to a prior art catheter assembly, namely the INSYTE™ AUTOGUARD™ BC Shielded IV Catheter available from Becton Dickinson & Company of Franklin Lakes, New Jersey. “IAG+H” refers to the prior art catheter assembly coupled to a flow restrictor, such as the flow restrictor 24 of FIGS. 1A-1B or the flow restrictor 38 of FIGS. 2A-2B. “UT” refers to another prior art catheter assembly. Hemoshield 1 was used in a hemolysis study with 24G IAG where all samples were hemolyzed (measured by the presence of plasma free hemoglobin) in the absence of a flow restrictor. With Hemoshield 1, hemolysis was reduced to the level of UT 21G.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A blood collection device, comprising:
a flow restrictor, comprising a distal end, a proximal end, and a fluid pathway, wherein the proximal end is configured to couple to a blood collection container, and wherein a geometric factor of the flow restrictor is represented by G_{f}, wherein G_{f} is at least one of:
(i) 1.43E7 1/in³±30%;
(ii) 3.70E6 1/in³±30%;
(iii) between 1.43E7 1/in³ and 3.70E6 1/in³;
(iv) 1.43E7 1/in³±10%; or
(v) 3.70E6 1/in³±10%.

2. The blood collection device of claim 1, wherein G_{f} is 1.43E7 1/in³±30%.

3. The blood collection device of claim 1, wherein G_{f} is 3.70E6 1/in³±30%.

4. The blood collection device of claim 1, wherein G_{f} is between 1.43E7 1/in³ and 3.70E6 1/in³.

5. The blood collection device of claim 1, wherein G_{f} is 1.43E7 1/in³±10%.

6. The blood collection device of claim 1, wherein G_{f} is 3.70E6 1/in³±10%.

7. The blood collection device of claim 1, wherein the blood collection container is a syringe.

8. The blood collection device of claim 1, wherein an overall length of the restrictor is less than an overall length of the syringe.

9. The blood collection device of claim 1, wherein the flow restrictor is monolithically formed as a single unit.

10. The blood collection device of claim 1, wherein the proximal end of the flow restrictor comprises a first luer adapter, the distal end comprises a second luer adapter, and wherein the flow restrictor further comprises an extension tube disposed between the first luer adapter and the second luer adapter.

11. The blood collection device of claim 1, wherein the distal end of the flow restrictor is configured to couple to a catheter assembly.

12. The blood collection device of claim 11, further comprising a 20 gauge (G) catheter coupled to the flow restrictor, wherein a length of the fluid pathway is represented by L, wherein a diameter of the fluid pathway is represented by D, wherein D⁴/L is equal to or less than 3.1E-07 cubic inches.

13. The blood collection device of claim 12, further comprising a catheter adapter, wherein the catheter extends distally from the catheter adapter.

14. The blood collection device of claim 11, further comprising a 22 gauge (G) catheter coupled to the flow restrictor, wherein a length of the fluid pathway is represented by L, wherein a diameter of the fluid pathway is represented by D, wherein D⁴/L is equal to or less than 7.0E-08 cubic inches.

15. The blood collection device of claim 14, further comprising a catheter adapter, wherein the catheter extends distally from the catheter adapter.

16. The blood collection device of claim 1, wherein the fluid pathway has a uniform diameter.

17. The blood collection device of claim 1, wherein the fluid pathway has a variable inner diameter along a length of the fluid pathway.
